(19) United States
(12) Patent Application Publication
(10) Pub. No.: US 2005/0101909 A1
(43) Pub. Date: May 12, 2005

(54) Apparatus for topical innoculation of pharmaceuticals

(76) Inventor: Cino Rossi, Rome (IT)

Correspondence Address: JORDAN AND HAMBURG LLP, 122 EAST 42ND STREET, SUITE 4000, NEW YORK, NY, 10168, US

(21) Appl. No.: 10/703,992

(22) Filed: Nov. 7, 2003

(51) Int. Cl. A61M 31/00

(52) U.S. Cl. 604/93.01; 604/96.01; 604/264

(57) ABSTRACT

Apparatus (1) for topical inoculation of pharmaceuticals comprises a tubular body (2), an inoculation needle (3) that can be inserted in said tubular body (2) in an axially guided sliding manner, at least one outlet opening (7) of a distal section of said inoculation needle (3) obtained in said tubular body (2), that is obtained laterally in said tubular body (2).

This invention concerns an apparatus for topical inoculation of pharmaceuticals.

In medical practice, in the presence of certain pathologies, it is sometimes necessary to resort to strictly topical inoculations of pharmaceuticals to carry out therapeutic interventions.

This may for example be necessary in gynaecology when medical operators have to intervene inside the female genital apparatus making small topical interventions that require the administration of an anaesthetic to make the patient temporarily desensitised.

Owing to the nature and configuration of the operating area, in order to be able to obtain said desensitisation, the patients have to be subjected to total anaesthesia, even if the intervention is per se simple.

This leads, even in those cases that are per se simple, to all the problems linked to the administration of anaesthetics, above all the compatibility with the physical conditions of the patients, even more so if they are affected by pathologies that make said administration of anaesthetics dangerous, all the more so if they are administered in significant doses.

The technical object of this invention is to eliminate the above disadvantages of the state of the art by excogitating an apparatus for topical inoculation of pharmaceuticals that enables said pharmaceuticals to be administered in a strictly accurate and focussed manner and therefore in doses that are strictly commensurate with the specific therapeutic needs.

These objects are all achieved by this apparatus for topical inoculation of pharmaceuticals as defined in claim 1.

Further characteristics and advantages of this invention will become clearer in the detailed description of a preferred but not exclusive embodiment of an apparatus for topical inoculation of pharmaceuticals illustrated by way of non-limiting example in the attached sheets of drawings wherein:

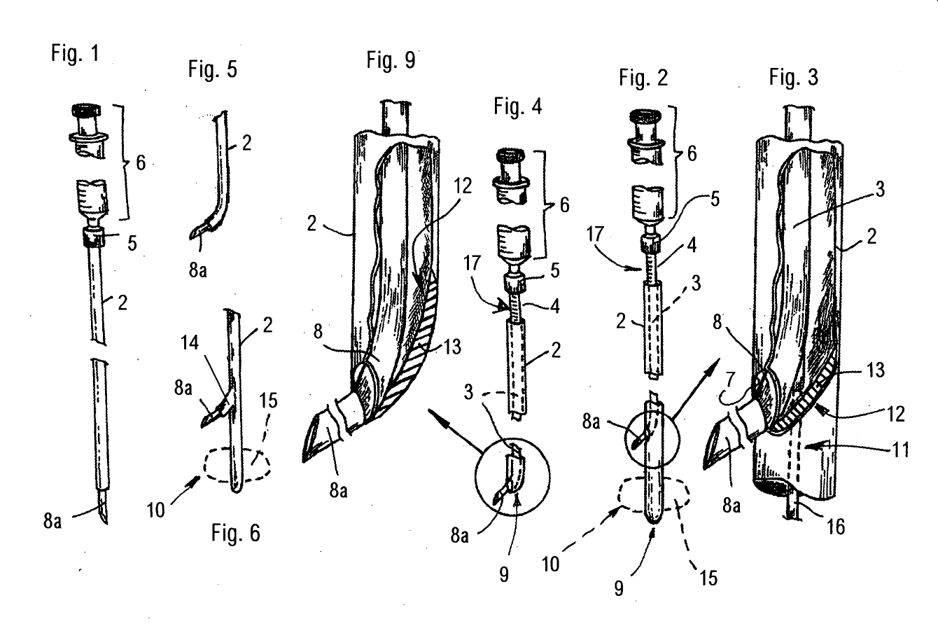

FIG. 1 is a general view of an apparatus known to the art for the topical administration of pharmaceuticals;

FIG. 2 is a general view of an apparatus for topical inoculation of pharmaceuticals, according to the invention;

FIG. 3 is an enlarged lateral view of a portion of the apparatus for topical inoculation of pharmaceuticals delimited by the circle in FIG. 2;

FIG. 4 is a general view of an apparatus for topical inoculation of pharmaceuticals in a second possible embodiment;

FIG. 5 also shows an apparatus for topical inoculation of pharmaceuticals according to the invention, in a third possible embodiment;

FIG. 6 is a view of a distal portion of a version of the third possible embodiment of the apparatus shown in FIG. 5;

FIG. 7 schematically shows an open portion of the female genital apparatus, in particular in the area of the uterus, showing use of the distal portion of the apparatus made according to the first embodiment of the invention illustrated in FIG. 1;

FIG. 8 also shows the same open portion of the female genital apparatus as FIG. 7, showing use of the distal portion of the apparatus made according to the third possible embodiment of the invention;

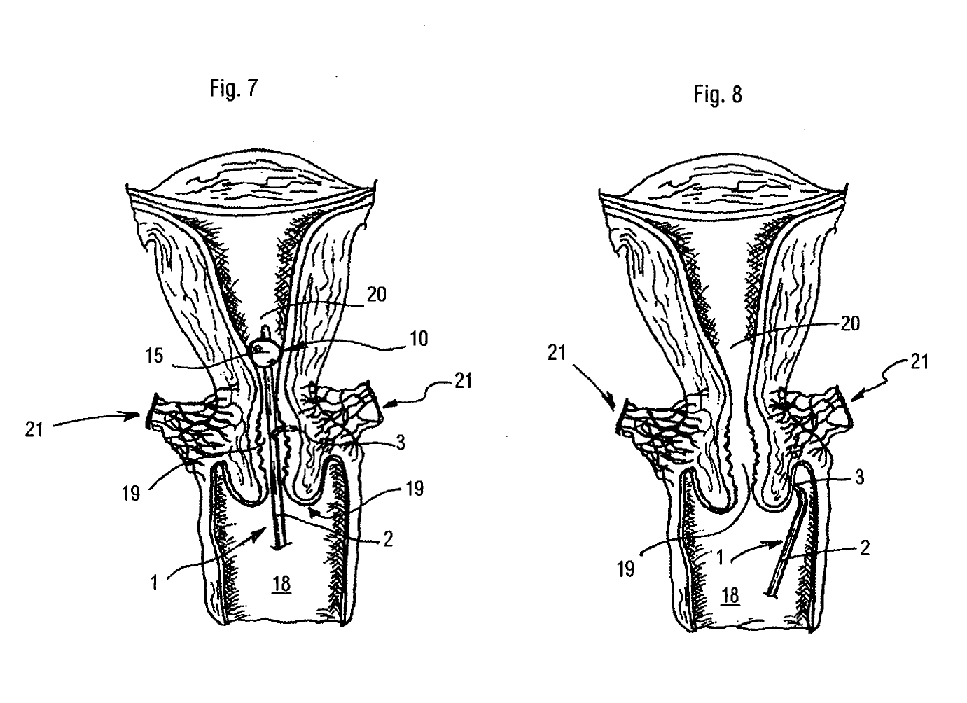

FIG. 9 shows on an enlarged scale the detail highlighted in the circle in FIG. 4;

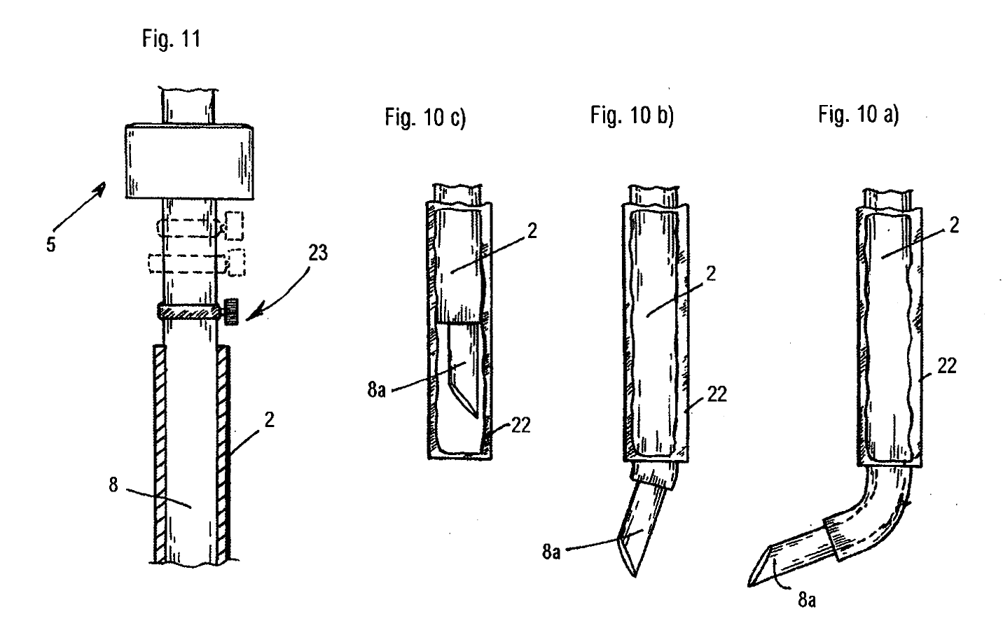

FIGS. 10a, 10b, 10c show a fourth possible embodiment of the apparatus for topical inoculation, according to the invention, equipped with a sliding external sheath, in three phases of use;

FIG. 11 shows a detail on an enlarged scale of a zone near an apparatus for topical inoculation according to the invention, provided with a means for locking sliding between inoculation needle and tubular body.

In the above figures, 1 indicates overall an apparatus for topical inoculation of pharmaceuticals that essentially comprises a substantially rigid tubular body 2, within which an inoculation needle 3 is slidingly inserted, which consists of a sort of flexible catheter provided with a rigid tip, which is axially guided and which, at the near end 4 normally facing the operators, has a standardised fitting 5 mounted upon it to which a pharmaceuticals feed means 6 can be attached, which preferably consists of a syringe.

In the tubular body 2 an opening 7 is obtained through which the distal end 8 of the inoculation needle 3 can emerge, i.e. the end equipped with a rigid tip 8a suitable for penetrating the bodily tissues.

Said outlet opening 7 is obtained laterally in said tubular body 2.

Said opening 7 can, in a first embodiment of the invention, be substantially provided on the distal end 9 of the tubular body 2 (see FIGS. 4 and 5), whereas, in a second embodiment, the opening 7 is provided at an intermediate height thereof and anyway near the distal end 9 (see FIGS. 2 and 6).

In said second embodiment, between the distal end 9 of said tubular body 2 and downstream of said outlet opening 7, an anti-slip element 10 can be mounted that can be commanded by a relative dilation means 11 to elastically dilate.

In said second embodiment, as the outlet opening 7 is obtained laterally in an intermediate position in the tubular body 2, the distal end 9 is blind.

Near the outlet opening 7 a deviation means 12 of said distal section 8 towards said outlet opening 7 is provided, which can be arranged inside the tubular body 2: in this case, said deviation means 12 comprises at least one curved diaphragm means 13 that is placed between said tubular body 2 and said outlet opening 7; said deviation means 12 can also be constituted by a tubular appendage 14 that extends laterally for a section protruding outside the outlet opening 7.

The elastically dilatable anti-slip element 10 consists of a sort of ball 15 that is fitted to the distal end of the tubular body 2 and which can be connected to the inside thereof.

The dilation means 11 comprises a channel 16 that is inserted inside the tubular body 2 and the distal end of which is connected to said sort of ball 15 and a means (not illustrated in the drawings) for feeding the dilating fluid that can be connected to the near end of the channel 16.

Between said tubular body 2 and said inoculation needle 3 a graduated mean 17 is placed to measure the sliding of said inoculation needle 3 in relation to the tubular body 2.

According to a third possible embodiment, the apparatus 1 for topical inoculation of pharmaceuticals also comprises a tubular body 2, an inoculation needle 3 that can be inserted in said tubular body 2 in an axially sliding manner, at least one outlet opening 7 of the distal end of said inoculation needle 3 obtained in said tubular body 2 the distal end 9 of which is laterally folded.

According to said third embodiment, at least the distal end 9 is made of stably formable (so-called memory material) and flexible material.

On the tubular body 2 a sheath 22 can also be fitted that slides thereupon, which is suitable for adopting two positions: the first raised (FIG. 10a) and a second lowered (FIG. 10c) on said distal end 9, until it is covered after being elastically lifted up sliding thereupon.

The use of the apparatus 1 for topical inoculation of pharmaceuticals is as follows: the tubular body 2 is inserted into the vaginal channel 18 of the patient in such a way that the distal end 9 reaches the neck 19 of the uterus 20.

During said insertion, the distal end of the inoculation needle 3 is kept retracted inside the tubular body 2 that constitutes the apparatus 1 and the anti-slip element 10, when provided, is kept deflated.

If the apparatus 1 is the rectilinear tubular body 2 embodiment and is equipped with a lateral opening 7 (see FIG. 7), the health worker proceeds by introducing the distal end 9 inside the neck 20 of the uterus, until the opening 7 is at the nerve bundle, indicated overall as 21, which has to be desensitised to be able to perform operations under local anaesthetic.

When the position of the apparatus 1 is defined, the health worker runs the inoculation needle 3 inside the tubular body 2, making the distal end 8 protrude by a few millimetres from said needle 3, i.e. the rigid inoculation tip 8a, from the opening 7 and locking the slide thereof, and therefore the position reached with the means 23 consisting for example of a collar clamp with locking screw (FIG. 11).

Said distal end of the inoculation needle 3 is deviated by flowing towards the outlet opening 7 from the diaphragm means 13 advantageously with a profile curved to constitute a directional guide; the opening 7 may also if necessary be equipped with a tubular appendage 14 that protrudes towards the exterior to continue the guide effect and reach the injection point with greater accuracy.

The sliding section of the inoculation needle 3 in relation to the tubular body 2 is constantly monitored by the health worker observing the graduated means 17.

When the inoculation needle 3 emerges from the outlet opening 7, it penetrates directly into the bodily tissues in the area near the nerve bundle 21 to be desensitised; an anaesthetic is then injected with a syringe or with a general feed means 6, that therefore reaches only the topical zone that surrounds the nerve bundles 21, thereby desensitising them.

If the medical worker deems its presence to be necessary, the tubular body 2 is equipped at the distal end 9 with an element 10 that can be commanded to elastically dilate, said element 10 in practice consisting of a sort of ball 15 that, when it is dilated inside the uterus 20 (FIG. 2), prevents the tubular body 2 accidentally slipping out through traction when the it is definitively positioned.

To reach the nerve bundle 21 it is also possible to use the second or the third embodiment of the apparatus 1, respectively illustrated in FIGS. 4 and 5.

In this case the correct positioning of the apparatus 1 for topical inoculation of pharmaceuticals is the one illustrated in FIG. 8 wherein the nerve bundle 21 is reached directly from the vaginal channel, using the lateral arrangement of the opening 7 at the distal end 9 of the tubular body 2 or the deviated curve of said distal end 9.

In order to be able to administer several inoculations in the affected zone, it is sufficient to rotate the tubular body 2 around its own longitudinal axis, having before any rotation retracted the inoculation needle 3 from the tubular body 2 and then pushing it towards the exterior without thereby removing the latter from the defined position.

On the tubular body 2, in the third possible embodiment of the apparatus 1 a sheath 22 can also be fitted that slides freely thereupon; at the same time, the tubular body 2 is made of elastic material equipped with ‘memory’, i.e. which can be stably preformed to create the lateral folding of the distal end 9, but can be elastically straightened by making the sheath 22 slide until it covers the tip 8a of the inoculation needle 8.

It is also possible, for certain types of intervention, for example to carry out electrocoagulations, to make the rigid tip 8a of the inoculation needle 3 out of an electrically conducive material and connect it to a source of electric energy of adequate intensity.

It has been established in practice that the disclosed invention achieves the proposed objects.

The invention that is thus conceived may undergo numerous modifications and embodiments, all of which fall within the scope of the protection as defined by the claims.

All details may be furthermore substituted by other technically equivalent details.

In practice, any materials, shapes and dimensions may be used without thereby falling outside the scope of protection of this invention.

1. Apparatus for topical inoculation of pharmaceuticals comprising:
a substantially rectilinear tubular body,
an inoculation needle that can be inserted in said tubular body in an axially guided sliding manner, and
at least one outlet opening for a distal section of said inoculation needle formed laterally in said tubular body.

2. Apparatus according to claim 1, wherein said outlet opening is at a distal end of said tubular body.

3. Apparatus according to claim 1, wherein said outlet opening is near a distal end of said tubular body.

4. Apparatus according to claim 1 or 3, further comprising an anti-slip element and a relative dilation means, said anti-slip element being mounted at said distal end of said tubular body and downstream of said outlet opening, the anti-slip element being elastically dilatable by a relative dilation means.

5. Apparatus according to claim 1 or 3, wherein said distal end of said tubular body is blind.

6. Apparatus according to any one of the claims 1 to further comprising near said outlet opening deviation means (12) of for deviating said distal section towards said outlet opening.

7. Apparatus according to claim 6, wherein said deviation means is arranged inside said tubular body and comprises at least one curved diaphragm means placed between said tubular body and said outlet opening.

8. Apparatus according to claim 6, wherein said deviation means (12) is comprised of a tubular appendage extending along a section protruding outside said outlet opening.

9. Apparatus according to claim 4, wherein said elastically dilatable anti-slip element (10) is comprised of an at least roughly spherical element fitted on said distal end of said tubular body and which can be connected with the inside thereof.

10. Apparatus according to claim 4 wherein said dilation means comprises:
at least one channel inserted in said tubular body and having a distal end connected to said element, and at least one dilating fluid means that can be connected to the end nearest said channel.

11. Apparatus according to any one of claims 1 to further comprising a graduated means between said tubular body and said inoculation needle, the graduated means measuring sliding of said inoculation needle in relation to said tubular body.

12. Apparatus according to any one of claims 1 to 3, further comprising a supply means of said inoculation needle the supply means being comprised of at least one syringe.

13. Apparatus according claim 12, further comprising, between said supply means and an end near said inoculation needle, a standardised fitting for reciprocal connection.

14. Apparatus according to any one of claims 1 to 3 wherein at least one said inoculation needle is made of electrically conductive material for connection with a source of electric energy.

15. Apparatus for topical inoculation of pharmaceuticals comprising:
a tubular body,
an inoculation needle that can be inserted in said tubular body in an axially sliding manner, and
at least one outlet opening for a distal end of said inoculation needle,
wherein at least a distal end of said tubular body is laterally deviated.

16. Apparatus according to claim 15, wherein at least said distal end of said tubular body is flexible.

17. Apparatus according to claim 15 or 16, further comprising a cylindrical sheath is slidingly fitted on said tubular body to cover or uncover said distal end of said tubular body.

18. Apparatus according to claim 15, wherein said outlet opening is at a distal end of said tubular body.

19. Apparatus according to any one of claims 1 to 3, 15, 16 and 18, further comprising, between said tubular body and said needle, locking means for preventing said sliding.

20. Apparatus according to claim 1 or 15, wherein said tubular body has a geometric transversal cross-section.

21. Apparatus according to claim 20, wherein said geometric transversal cross-section is a substantially ellipitical cross-section.
